(19) United States
(12) Patent Application Publication
(10) Pub. No.: US 2014/0119992 A1
(43) Pub. Date: May 1, 2014

(54) METHOD, COMPOSITION, AND SYSTEM FOR DISINFECTION

(71) Applicant: Nevoa Life Sciences, Scottsdale, AZ (US)

(72) Inventor: Emmett Manuel Cunningham, Nags Head, NC (US)

(73) Assignee: Nevoa Life Sciences, Scottsdale, AZ (US)

(21) Appl. No.: 14/067,364

(22) Filed: Oct. 30, 2013

Related U.S. Application Data
(60) Provisional application No. 61/720,442, filed on Oct. 31, 2012.

(51) Int. Cl. A01N 59/00; A01N 25/06; A61L 2/22; A01N 31/02

(52) U.S. Cl. CPC A01N 59/00; A01N 31/02; A01N 25/06; A61L 2/22
USPC 422/37; 424/661

(57) ABSTRACT

Embodiments of the invention provide methods, compositions, and systems for disinfection. In one embodiment, the invention provides a disinfecting solution comprising: a liquid having an HOCl concentration between about 50 ppm and about 4000 ppm; and a quantity of ethyl alcohol comprising between about 0.05% and about 10% of the disinfecting solution, by weight.

### CROSS-REFERENCE TO RELATED APPLICATIONS

This application claims the benefit of co-pending U.S. Provisional Patent Application No. 61/720,442, filed 31 Oct. 2012, which is hereby incorporated herein.

Embodiments of the present invention relate methods, compositions, and systems capable of ridding populous areas of fungal, bacterial, and viral infectious agents. This not only includes schoolrooms, gyms and showers rooms, but airplanes, cruise ships, veterinary clinics, and, especially, hospitals and their operating rooms, but any area inhabited by humans or animals. Today there are chemical based and non-chemical based solutions comprising aromatic, sprayed, and wipe on products, some of which are hazardous.

Methods according to some embodiments of the invention use a fogging delivery system that will deliver a Super-Anolyte Micro Fog consisting of a solution of Anolyte having a HOCl content of 50-4000 ppm and a volume of alcohol (ethyl alcohol), e.g., at 190 proof, between 0.05% and 10%, by weight. The addition of a small amount of alcohol increases the killing capabilities of the Anolyte solution by a factor of 5-10 as compared to similar strength Anolyte without the additional alcohol. If too much alcohol is added to the base liquid of Anolyte it will alleviate all free available chlorine, (FAC), and render the solution ineffective. The Super-Anolyte solution should be used within 48 hours of preparation, as the relative proportion of HOCL dissipates very rapidly.

This combination of elements, Anolyte and alcohol, is distinctly different from all the types available to the disinfecting market today including all forms of Anolyte. This invention would incorporate the latest technology in the field of micro fogging including, but not limited to, rotary disc, high-pressure nozzles, and piezoelectric ultrasonic atomizing. The use of these technologies allows the invention to be produced as a “Micro Fog of Super-Anolyte” (“MFSA”) in the 1-50 micron droplets. This is very important for two reasons; one, this micron size allows the MFSA to disperse to all areas of the treated area (under, around and in everything), and two, this micron size allows the MFSA to attack every known infectious agent no matter what size or location, killing it on contact or within minutes. The Super-Anolyte can also be directly sprayed onto any surface by the use of any standard trigger or pump sprayer on the market. After it is allowed to work for a few minutes it would be wiped down in the same manner as any other disinfectant. This also could be used directly on food preparation area without harm to humans or animals.

Anolytes such as those suitable for use according to the invention have been well documented in their ability to completely destroy virtually all bacteria, fungi, and viruses within minutes, including methicillin-resistant Staphylococcus aureus (MRSA) and Clostridium difficile (C. diff). One such anolyte is an Electro-Chemical Activation (ECA Technology) process of water generated by electrolysis of a dilute NaCl solution passing through an electrolytic cell. Besides being super-oxidized, it contains hypochlorous acid (HClO) and hypochlorite ion (OCl⁻) which give it superior sporicidal and biocidal activity. Such an anolyte has further been tested to show there is no known effect on the epidermis, dermis, or the fatty tissue after extended contact. The addition of a very small amount of pure grain alcohol serves as a catalyst or stimulant to the kill rate of the anolyte without having any detrimental effect on the human or animal inhabitants of the area. By using a MFSA system in the 1-50 micron size, the treated area will not retain any moisture on its surface or contents that could induce mold or areas that would propagate any form of microbe in the near future.

The MFSA system super-anolyte permeates the area so quickly, with its higher kill rate, that the system only needs to operate for short periods of time therefore reducing the amount of liquid needed for decontamination. This allows for immediate reuse of the area and will not harm humans or animals. The fine mist does not “wet-down” the area so as to require further cleaning or wiping. The MFSA system virtually eliminates the need for chemical wipe down decontamination of the area after fogging. An area could be totally decontaminated in 10-15 minutes and ready for reuse.

What is claimed is:

1. A disinfecting solution comprising:
a liquid having an HOCl concentration between about 50 ppm and about 4000 ppm; and
a quantity of ethyl alcohol comprising between about 0.05% and about 10% of the disinfecting solution, by weight.

2. The disinfecting solution of claim 1, wherein the ethyl alcohol comprises between about 0.1% and about 7% of the disinfecting solution, by weight.

3. The disinfecting solution of claim 2, wherein the ethyl alcohol comprises between about 0.5% and about 5% of the disinfecting solution, by weight.

4. The disinfecting solution of claim 3, wherein the ethyl alcohol comprises between about 0.5% and about 2% of the disinfecting solution, by weight.

5. The disinfecting solution of claim 3, wherein the ethyl alcohol comprises between about 1% and about 3% of the disinfecting solution, by weight.

6. The disinfecting solution of claim 5, wherein the ethyl alcohol comprises between about 2% and about 3% of the disinfecting solution, by weight.

7. A method of disinfecting an area, the method comprising:
producing, within an area to be disinfected, a fog of a liquid disinfectant, the fog comprising liquid droplets between about 1 μm and about 50 μm in size,
wherein the liquid disinfectant includes a liquid having an HOCl concentration between about 50 ppm and about 4000 ppm and a quantity of ethyl alcohol comprising between about 0.05% and about 10% of the disinfecting solution, by weight.

8. The method of claim 7, wherein the liquid droplets are between about 1 μm and about 15 μm in size.

9. The method of claim 8, wherein producing includes producing the fog using at least one one device selected from a group consisting of: a high-speed rotary disc, a nozzle, and a piezoelectric ultrasonic atomizer.

10. The method of claim 8, wherein the producing is delayed to permit evacuation of the area.

11. The method of claim 8, wherein the producing is timed to continue for a pre-determined length of time.

12. The method of claim 8, wherein the producing is timed to automatically end after a pre-determined length of time.

13. A disinfecting composition comprising:
a liquid having an HOCl concentration between about 50 ppm and about 4000 ppm.

14. The disinfecting composition of claim 13, wherein the composition is in the form of a fog comprising liquid droplets between about 1 μm and about 50 μm in size.

15. The disinfection composition of claim 14, wherein the liquid droplets are between about 1 μm and about 15 μm in size.
